(19) United States
(12) Patent Application Publication
(10) Pub. No.: US 2008/0166720 A1
(43) Pub. Date: Jul. 10, 2008

(54) METHOD AND APPARATUS FOR RAPID NUCLEIC ACID ANALYSIS

(75) Inventors: Albert Tsung-Hsi Hsieh, Cypress, CA (US); Abraham P. Lee, Irvine, CA (US)

Correspondence Address: Vista IP Law Group LLP, 2040 MAIN STREET, 9TH FLOOR, IRVINE, CA, 92614, US

(73) Assignee: THE REGENTS OF THE UNIVERSITY OF CALIFORNIA, Oakland, CA (US)

(21) Appl. No.: 11/868,404

(22) Filed: Oct. 5, 2007

Related U.S. Application Data
(60) Provisional application No. 60/828,542, filed on Oct. 6, 2006.

(51) Int. Cl. C12Q 1/68

(52) U.S. Cl. 435/6; 435/287.2

(57) ABSTRACT

A system for detecting the presence of a target nucleic acid sequence in a sample includes a microfluidic device having a droplet generation region for forming a plurality of droplets, the droplet generation region being operatively coupled to a sample inlet coupled to a sample source, a molecular beacon inlet coupled to a source of molecular beacon material configured to hybridize to the target nucleic acid sequence, and a source of carrier material on opposing sides of the droplet generation region. Each droplet generated in the microfluidic device includes sample material and molecular beacon material. After generation, the droplets pass to a downstream mixing channel coupled to the droplet generation region. The system further includes an optical system configured to detect fluorescent emissions from droplets flowing in the mixing channel The presence of fluorescence is indicative of the presence of the target nucleic acid sequence within the sample.

### RELATED APPLICATION

This application claims priority to U.S. Provisional Patent Application No. 60/828,542 filed on Oct. 6, 2006. Priority is claimed pursuant to 35 U.S.C. § 119. The above-noted application is incorporated by reference as if set forth fully herein.

### STATEMENT REGARDING FEDERALLY SPONSORED RESEARCH AND DEVELOPMENT

This invention was made, in part, with Government support of Grant No. NAS2-03144 awarded by the National Aeronautics and Space Administration (NASA). The Government may have certain rights in this invention.

### FIELD OF THE INVENTION

The field of the invention generally relates to microfluidic devices. More particularly, the field of the invention relates to microfluidic devices capable of using a molecular beacon in small (e.g., picoliter) sized droplets for nucleic acid and protein detection and binding kinetic analysis.

### BACKGROUND

Breast cancer is a world wide public health concern and the estimated death toll in the United States in 2001 was approximately 40,000. Breast cancer is also ranked first as the cause of cancer death for women between ages 20 to 59 years. People carrying mutation in both the BRCA1 and BRCA2 genes are prone to breast cancer and ovarian cancer. The conventional DNA detection method which involves a heterogeneous solid-liquid hybridization process requires probe immobilization. Stringent rinsing to remove non-specific DNA bonding is required and the entire process is time-consuming and also requires a significant number of samples.

A new technique has been developed that utilizes molecular beacons (MB or MBs). MBs are one class of Fluorescence Resonance Energy Transfer (FRET) molecules and have been reported for the construction of probes that are useful for real-time detection of nucleic acids. MBs are synthesized as single stranded nucleic acid molecules that are constructed by stem and loop structures. The loop portion contains sequences complementary to the target single stranded DNA. The stem portion is formed by annealing two complementary arm sequences that are not related to the target single strand DNA. MB becomes fluorescent only when the probes encounter the target single strand DNA. MB also has outstanding capability for the selective detection of single nucleotide polymorphisms (SNPs).

Conventionally, MB hybridizes with a target gene or genetic sequence merely by diffusion (i.e., a concentration gradient). It generally takes at least 10 minutes or up to 6 hours in a liquid-liquid environment to complete the entire hybridization process. Other traditional DNA detection techniques that use DNA probe immobilization (e.g., liquid-to-solid hybridization) require time consuming stringent rinsing and gel electrophoresis and stringent rinsing. This entire process may take over 12 hours to complete.

There is a need for a method and device that can use MBs to rapidly detect or sense a target nucleic acid sequence or protein. The nucleic acid sequence may include a gene or genetic sequence. Such a device and method would enable rapid turn-around of laboratory analysis results. For example, in the context of the BRCA1 gene, a patient sample could be taken and analyzed in a matter of minutes or even seconds.

### SUMMARY

According to one embodiment of the invention, a system for detecting the presence of a target nucleic acid sequence in a sample includes a microfluidic device having a droplet generation region for forming a plurality of droplets, the droplet generation region being operatively coupled to a sample inlet coupled to or containing a sample source, a molecular beacon inlet coupled to or containing a source of molecular beacon material configured to hybridize to the target nucleic acid sequence, and a source of carrier material on opposing sides of the droplet generation region. Relatively small (e.g., picoliter) sized droplets are pinched-off in the droplet generation region by the side streams of the carrier material which may comprise an oil. Thus, each droplet generated in the microfluidic device includes sample material and molecular beacon material. After generation, the droplets pass to a downstream mixing channel coupled to the droplet generation region. The mixing channel may include a number of substantially 90° turns to give the mixing channel a sawtooth configuration. Alternatively, the mixing channel may be substantially straight. The system further includes an optical system configured to detect fluorescent emissions from droplets flowing in the mixing channel. The presence of fluorescence is indicative of the presence of the target nucleic acid sequence.

In another embodiment of the invention, a microfluidic device includes a substrate having a first inlet disposed in the substrate and coupled to first and second channels terminating on opposing sides of a junction. The first inlet may be adapted to contain a carrier material such as oil. Second and third inlets are disposed in the substrate and are both coupled to respective channels that terminate at an upstream side of the junction. One of the second and third inlets is adapted to contain a sample material while the other inlet is adapted to contain molecular beacon material. A mixing channel is coupled to a downstream side of the junction and includes a plurality of substantially 90° turns to give the mixing channel a sawtooth configuration.

In still another embodiment of the invention, a method of detecting the presence of a target nucleic acid sequence in a sample includes providing a microfluidic device having a mixing channel coupled to an output of a droplet generation region, the droplet generation region being coupled to first and second channels adapted to contain an oil phase and first and second reactant inlets, wherein one of the first and second reactant inlets is coupled to a sample and the other of the first and second reactant inlets is coupled to a source of molecular beacon material. The first and second reactants along with the oil phase are then pumped or otherwise flowed into the droplet generation region so as to generate a plurality of droplets in the generation zone. The fluorescence level of the droplets in at least a portion of the mixing channel is monitored via an optical system, wherein the fluorescence level is indicative of the presence of the target nucleic acid sequence in the sample.

In another embodiment, a method of monitoring the binding kinetics of an analyte and a molecular beacon includes providing a microfluidic device having a mixing channel coupled to an output of a droplet generation region, the droplet generation region being coupled to first and second channels adapted to contain an oil phase and first and second reactant inlets, wherein one of the first and second reactant inlets is coupled to a source of analyte and the other of the first and second reactant inlets is coupled to a source of molecular beacon material. The first and second reactants and the oil phase are pumped or otherwise flowed into the droplet generation region so as to generate a plurality of droplets in the generation zone. The fluorescence level of the droplets is monitored in at least a portion of the mixing channel, wherein the fluorescence level at a given point in the mixing channel is indicative of the binding kinetics between the analyte and the molecular beacon.

### BRIEF DESCRIPTION OF THE DRAWINGS

FIG. 1 schematically illustrates a microfluidic device used to generate microdroplets for mixing MBs and nucleic acids according to one embodiment.

FIG. 2 illustrates a schematic representation of a fluorescence analysis system according to one embodiment.

FIG. 3 is a photographic image of the droplet generation region according to one aspect of the invention.

FIG. 4 illustrates the threshold fluorescence image of fluorescent droplets moving in the sawtooth-shaped microchannel of the device illustrated in FIG. 1.

FIG. 5 is a high-speed photographic image of droplets in substantially 90° turns within the sawtooth-shaped microchannel of the device illustrated in FIG. 1.

FIG. 6 illustrates advection and bulk flow lines inside and around the droplets while moving in the mixing channel portion of the device.

FIG. 7 is a graph illustrating the fluorescence intensity versus time after droplets are generated. The fluorescence intensity of FRET molecules in micro droplets gradually increases along the channel from pinch-off point to the reservoir.

FIG. 8 illustrates the construction of an exemplary MB molecule. The MB molecule includes loop and stem portions. The loop portion contains the complementary nucleic acid sequence to the target nucleic acid (e.g., ssDNA). The stem portion contains complementary sequences to maintain the MB molecule closed in the absence of the target sequence.

### DETAILED DESCRIPTION OF THE ILLUSTRATED EMBODIMENTS

According to one aspect of the invention, a microfluidic-based analysis system 10 is provided that is a promising reactor for biological and chemical assays. The analysis system 10 offers numerous advantages including the ability to greatly reduce reaction time. In addition, the chemical concentration in each droplet 60 can be precisely controlled. Further, only a small amount of sample (and reagents) are needed for testing. The fluorescent droplet signal may be amplified by accumulated fluorescence from repeating “chemically identical droplets” in a period of exposure time. Many advantages for using droplet based devices have recently been reported, including the rapid mixing of liquids that are normally hindered in low Reynolds number single phase laminar flow. The strong convection inside the droplet 60 accelerates nucleic acid detection when using free solution molecular beacons (MB) bioassays making it much faster than conventional time-consuming methods such as heterogeneous solid-liquid hybridization process.

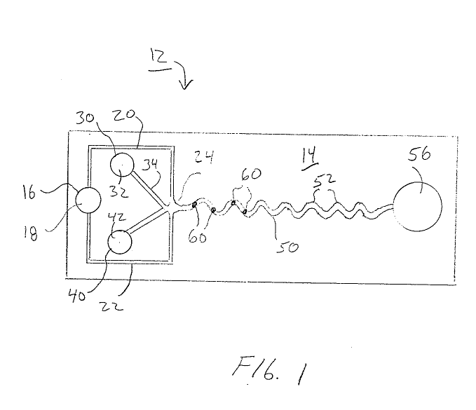

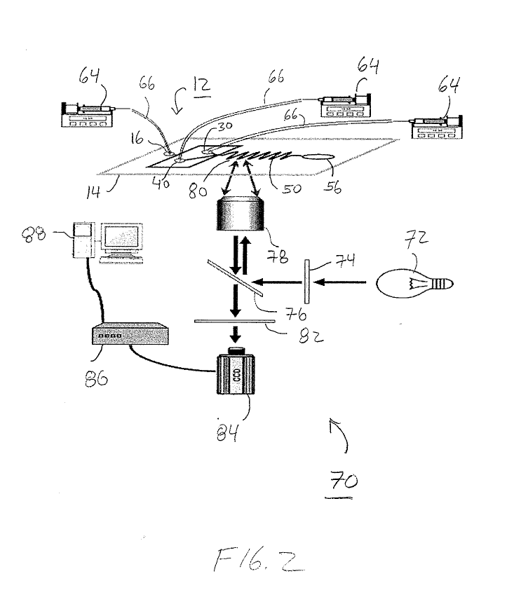

FIG. 1 illustrates a microfluidic device 12 that is used in connection with the analysis system 10 (illustrated in FIG. 2). The microfluidic device 12 includes a substrate 14 on which the various microfluidic features are located. The substrate 14 may be created from a substantially chemically-inert material. For example, the substrate 14 may be formed, in part, from a polymer such as polydimethylsiloxane (PDMS). PDMS allows for the relative easy formation and release of substrates 14 having various microfluidic features from a reusable SU-8 epoxy master. Details on the use of PDMS to form microfluidic structures may be found in D. C. Duffy et al., Rapid Prototyping of Microfluidic Systems in Poly(dimethylsiloxane), Anal. Chem., 70 (23), 4974-4984, 1998, which is incorporated by reference as if set forth fully herein. The microfluidic features can be made by pattern molds formed on a silicon wafer using SU-8 epoxy based negative photoresist (MicroChem, Newton, Mass.). PDMS (Sylgard 184, Dow Corning, MI) may be cured on the SU-8 mold in a 65° C. oven for one hour. The PDMS channel can be released from the SU-8 mold and sealed to a glass slide surface (forming a top) permanently after both being treated by oxygen plasma. The PDMS microchannel may then be incubated in 120° C. oven for 72 hours to convert microchannel surfaces from hydrophilic to hydrophobic. The microfluidic features may include inlets, channels, junctions, reservoirs and the like. PDMS is also advantageous because it has excellent transparent optical properties and does not posses any auto-fluorescence properties.

Still referring to FIG. 1, the substrate 14 includes a first inlet 16 that is configured to contain a carrier material 18 for the droplets 60. Generally, the carrier material 18 may include an immiscible continuous phase material such as, for instance, oil. One example of such oil includes silicon oil. The first inlet 16 is fluidicially coupled to two separate channels 20, 22 that terminate in a junction or droplet generation region 24. As explained below, the droplet generation region 24 includes a pinch-off area or region that “pinches-off” droplets generated from the streams flowing from the second inlet 30 and third inlet 40. The channel width of the droplet generation region 24 may be on the order of about 30 μm. The second inlet 30 is configured to contain a sample material 32. The sample material 32 may include a nucleic acid sequence such as a gene or gene sequence. The device 10 is used to detect the presence of target nucleic acid within the sample 32. The target nucleic acid may be indicative or correlated with a disease state such as, for example, cancer. For example, the target may include ssDNA from the BRCA1 or BRCA2 gene.

Experiments were conducted (described below) in which the sample material 32 included ssDNA-SynBRCA1 synthesized as portion of the target ssDNA from a 21 nucleotide (nt) portion sequence of the BRCA1 gene (thus the sample material 32 was the target nucleic acid). The sequence of SynBRCA1 (available from Integrated DNA Technologies, Inc.) is 5′-TAAC-ACAACAAAGAGC-ATACATAGG-GTTT-3′. It is a 29-nt oligonucleotide complementary to the middle 21 nucleotides of the MB's loop portion. This “test” target material was used to judge the effectiveness of the system 10 in identifying fluorescent radiation emitted when complementary sequences of nucleic acid were present within the formed droplets 60. During practical use of the system 10, the sample material 32 would include nucleic acid obtained from, for example, a subject patient that is being tested. For example, biopsy tissue from the patient may be collected, processed, and nucleic acid may be extracted for analysis on the system 10.

Referring back to FIG. 1, the second inlet 30 is fluidically coupled to a channel 34 that terminates at the droplet generation region 24. The third inlet 40 is configured to contain a MB 42. Generally, the MB 42 is a synthesized oligonucleotide which has a fluorophor label 106 at one end and quencher 108 at the other end. FIG. 8 illustrates the construction of a MB 42 molecule according to one aspect of the invention. The MB 42 includes a loop portion 100, a stem portion 102, and linkers 104 that connect respective ends of the nucleotide sequence to a fluorophor 106 and quencher 108. The loop portion 100 of the MB 42 includes the specific or complementary sequence that hybridizes with the target nucleic acid sequence. The MB 42 becomes fluorescent when it encounters the target nucleic acid sequence (e.g., gene or gene sequence). When the target nucleic acid is present and hybridizes with the complementary sequence in the loop portion 100, the MB molecule opens and the quencher 108 no longer prevents fluorescence. In response to excitation, fluorescent radiation is emitted which, as explained below, may be analyzed to detect the presence of the target nucleic acid in the sample 32.

With respect to identification of the BRCA1 gene described above, the MB 42 has the complementary sequence to the 21-nt of SynBRCA1 in the loop portion. The MB 42 was synthesized with Cy3 labeled at its 5′-terminus as the fluorophor and Black-Hole-Quencher2 (BHQ2) as quencher couple to its 3′ terminus as the quencher. The designed and synthesized MB is composed of 35 nucleotides. It has 21-nt loop and a 7-bp stem constituted by two 7-nt complementary arm sequences. The sequence of MB is Cy3-5′-CCTAGCC-CCTATGTATGCTCTTTGTTGT-GGCTAGG-3-BHQ2 (available from Integrated DNA Technologies, Inc.). The underlined nucleotide sequences disclosed above are complimentary to one another. Still referring to FIG. 1, the third inlet 40 is fluidically coupled to a channel 44 that terminates at the droplet generation region 24.

A mixing channel 50 is located downstream of the droplet generation region 24. The mixing channel 50 is where the sample material 32 and MB 42 are mixed in the generated droplets 60. The mixing channel 50 may have a substantially straight configuration or, alternatively, as illustrated in FIG. 1, the mixing channel 50 may have an undulating or sawtooth configuration. The sawtooth configuration generates advection flow in individual moving droplets 60 to improve mixing efficiency. In the sawtooth configuration, the undulating patterns are made by a plurality of substantially 90° turns 52. The mixing channel 50 may have a length on the order of around 20 mm, a width of 75 μm, and a height of 50 μm although other dimensions are intended to fall within the scope of the invention. The other channels, e.g., channels 20, 22, 34, and 44 may have similar widths and heights. Still referring to FIG. 1, the mixing channel 50 terminates into a product reservoir 56. The product reservoir 56 may include a chamber or the like that is used to accumulate carrier material 18 and droplets 60. As explained below, this product reservoir 56 may be periodically or continuously evacuated to avoid filling. Alternatively, the product reservoir 56 may be dimensioned such that it will not fill if the device 12 is run in batch mode.

FIG. 2 schematically illustrates a system 10 for the analysis and detection of nucleic acid according to one embodiment of the invention. As seen in FIG. 2, the microfluidic device 12 is coupled to pumps 64 via tubing 66. For example, separate pumps 64 may be coupled to the first inlet 16, second inlet 30, and third inlet 40. The pumps 64 may include syringe pumps available from Harvard Apparatus, MA. For example, the carrier material 18 may be pumped at a rate of around 10 μL/minute while both the sample material 32 and the MB 42 are pumped at a slower rate of 2 μL/minute. As the streams from the second and third inlets 30, 40 enter the droplet generation region 24 (i.e., junction point), they merge into a main stream. The merged main stream is then immediately pinched-off by the shear force from the side streams of carrier material 18 and form monodisperse, picoliter-sized droplets 60.

As seen in FIG. 2, an optical system 70 is provided to detect and/or measure fluorescence of the droplets 60 as they pass down the mixing channel 50. The optical system 70 includes an illumination source 72 which may include a halogen lamp which passes through an excitation filter 74 (available from Chroma, VT). The filtered light then is reflected off a beam splitter mirror 76 (available from Chroma) and passes to a 20× objective lens 78 (Plan Fluorite, Nikon USA). The objective lens 78 focuses illumination light onto a detection window 80 located within the mixing channel 50. Fluorescent light is emitted when the nucleic acid from the sample material 32 matches the complementary sequence of the loop portion of the MB 42. When this happens, the stem portion of the MB 42 will separate and the fluorophor 106 will emit fluorescence. This fluorescence is then collected by the same 20× objective lens 78 and passes through the beam splitter mirror 76 and through another emission filter 82. A camera 84 is used to capture image(s) of the emitted fluorescent light. For example, the camera 84 may include a monochrome high resolution CCD camera suitable for high resolution fluorescence imaging (Hamamatsu USA, NJ). The camera 84 is operatively coupled to a CCD camera controller and signal processing circuitry 86 (Hamamatsu USA, NJ). The system 70 may includes a separate computer 88 which contains fluorescence analysis software for the manipulation, analysis, and storage of data.

In one embodiment, DNA, RNA, protein, cell or other nucleic acid material of the subject may collected and be partially digested using enzymes or the like and run through the microfluidic device 12 for testing. For example, the sample material 32 may be loaded or pumped into the second inlet 30. In this regard, there is no synthetic oligomer formed—the patient's actually DNA (or other nucleic acid) is used for analysis. When protein is used as the target material, the MB 42 comprises a modified aptamer. Examples of fluorescent aptamer probes may be found in Fang et al., Molecular Aptamer for Real-Time Oncoprotein Platlet-Derived Growth Factor Monitoring by Fluorescence Anisotropy, Anal. Chem. 2001, 73, 5752-5757, which is incorporated by reference. In regards to DNA-protein interactions, MB 42 such as that disclosed in Li et al., Molecular Beacons: A Novel Approach to Detect Protein-DNA Interactions, Angew. Chem. Int. Ed. 2000, 39, No. 6, 1049-1052, which is also incorporated by reference, may be employed. Cells may even be encapsulated within the droplets 60 as explained in Tan et al., Controlled Microfluidic Encapsulation of Cells, Proteins, and Microbeads in Lipid Vesicles, J. Am. Chem. Soc. 2006, 128, 5656-5658 (2006), which is also incorporated by reference.

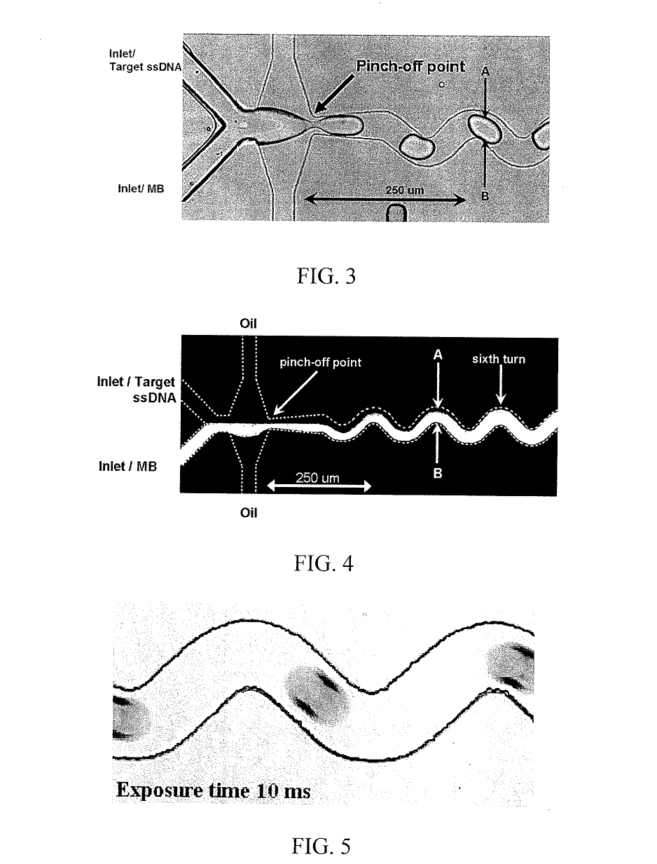

FIG. 3 illustrates a photographic image of the droplet generation region 24 of the microfluidic device 12. Images were taken using a monochrome high-speed camera (not shown in FIG. 2) that was used to observe the generation and velocity of the droplets 60. As seen in FIG. 3, emulsion monodisperse droplets 60 having picoliter volumes were generated. The width of the microfluidic channel was 71.6 μm, the height was 50 μm, and the length was 20 mm. Droplets 60 are illustrated being pinched-off from the pinch-off point in the droplet generation region 24.

FIG. 4 illustrates the threshold fluorescence image of fluorescent droplets 60 moving in the sawtooth-shaped mixing channel 50 of the microfluidic device 12. In this configuration, SynBRCA1 was loaded into the device 12 via the second inlet 30 and MB-BRCA1 was loaded into the device 12 via the third inlet 40. As seen in FIG. 4, the upper (or left) part of mixing channel 50 is dark while the fluorescence of the MB 42 remains in the bottom (or right) part of the mixing channel 50 at the pinch-off point. As the droplets 60 continue down the mixing channel 50, the fluorescence of MB 42 starts to shift to upper half of mixing channel 50 at the first 90° turn 52. After the sixth turn or switchback of the mixing channel 50, the fluorescence of MB 42 fills the entire droplet 60 and the dark region of droplet 60 in the downstream portion of the mixing channel 50 disappears. Because the design of the sawtooth-shaped mixing channel 50 has symmetric turns 52, once the MB 42 shift from lower part of droplet 60 to upper part, the nucleic acids also shift from lower part to upper part. Not only does the fluorescence of MB-BRCA1 fill the whole droplet 60, so does the SynBRCA1 fill whole droplet 60. It is thus confirmed that there is rapid mixing inside the microdroplets 60. The sawtooth geometry in the mixing channel 50 induced chaotic advection which rapidly mixed MB-BRCA1 and SynBRCA1 within the droplets 60. Because the length from the pinch-off point to the sixth turn corner is around 606 μm, and the droplet velocity was measured at around 8625 μm sec⁻¹, the mixing completion time is approximately 70 milliseconds.

The MB-BRCA1 hybridizes to SynBRCA1 virtually instantly once mixing starts. The volume of droplet 60 is around 238.5 picoliter and the droplet 60 is rapidly and thoroughly mixed at the sixth turn 52 in the mixing channel 50. Because of the geometry of the mixing channel 50, the thickness of the striation layer or diffusion layer is much reduced. The mixing and hybridization of MB 42 and ssDNA are based on the mixing flows in the droplets 60 and not merely rely on the diffusion of those molecules.

The fluorescence of MB 42 in the mixing channel 50 increases along the channel length from the pinch-off point (distance=0) to the product reservoir 56 after the BRCA1 hybridizes with MB, as indicated in FIG. 4. FIG. 5 illustrates images taken from a CCD camera with exposure time of 10 milliseconds of droplets 60 passing through turns 52 of the mixing channel 50. The measured displacement of the droplets 60 is 62.2 μm. The velocity of droplets in the channel is thus 6.22 mm/sec. Because the total length of the mixing channel 50 is 15.83 mm, it takes only 2.54 seconds for a droplet 60 to flow through the mixing channel 50 after its generation. Assuming that all the MBs 42 thoroughly hybridized with its complementary nucleic acid sequences (e.g., genes) in the droplets 60 before reaching the product reservoir 56, the hybridization rate will be less than 2.54 sec. This hybridization time is much faster as compared with conventional solid-to-liquid DNA hybridization which requires at least six hours for the entire process. Consequently, the detection method described herein is much faster.

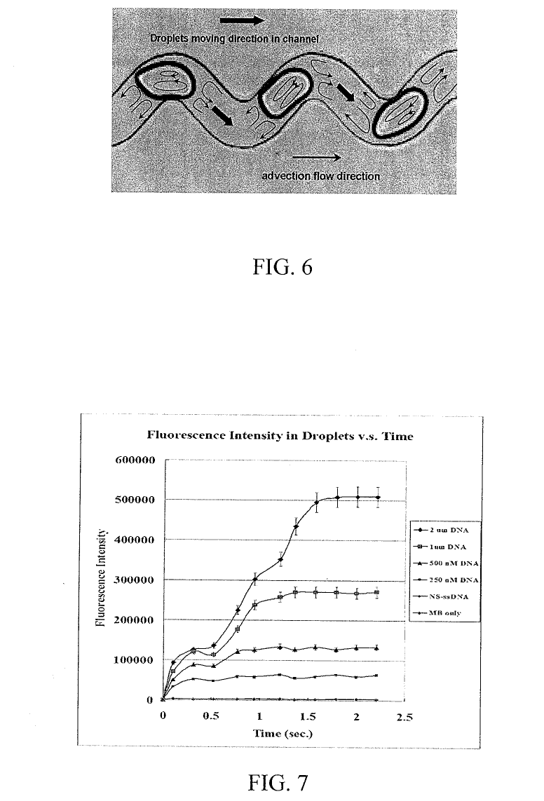

FIG. 6 illustrates how the sawtooth configuration of the mixing channel 50 generates advection flow in individual moving droplets 60 to improve mixing efficiency. The bulk flow of the droplets 60 within the mixing channel 50 is illustrated (directed to the right in FIG. 6) as well as the convection created within the droplets 60. The chaotic advection mixing of the droplets 60 is induced by the winding or undulating nature of the mixing channel 50. It is this advection mixing which ensures that the MB 42 and target nucleic acid are mixed within the first few turns 52 of the mixing channel 50.

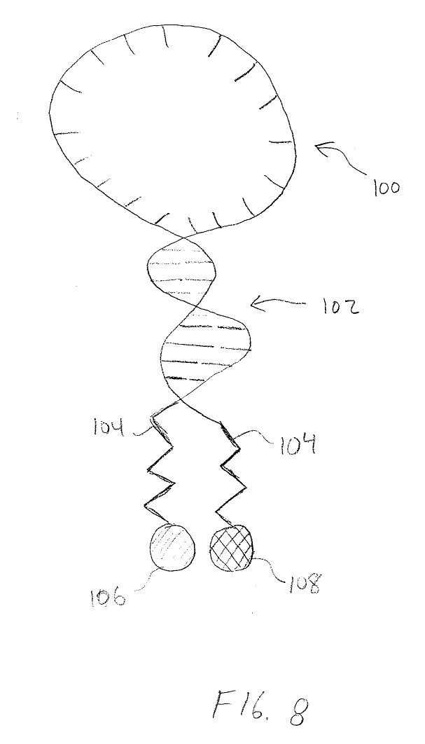

FIG. 7 illustrates the measured fluorescent intensity as a function of time for droplets 60 generated in the microfluidic device 12 for different concentrations of target ssDNA and non-specific DNA. As seen in FIG. 7, the fluorescent intensity of the FRET molecules inside the droplets 60 progressively increases along the mixing channel 50 from the pinch-off point to the downstream product reservoir 56. Moreover, the observed fluorescence intensity for 2 μM of MB-BRCA1 molecules hybridized with 2 μM of SynBRCA1 in droplets 60 reached the intensity plateau or saturation region after about two seconds. Thus, the 2 μM of MB-BRCA1 molecules hybridized with all the 2 μM SynBRCA1 molecules in droplets 60 within about two seconds. Similarly, the 1 μM of SynBRCA1 in droplets 60 can be detected in 1.5 seconds, and the detection of 250 nM SynBRCA1 in droplets 60 can be completed in less than 250 milliseconds. The lower the concentration of target ssDNA in the droplets 60 or the higher MB to target nucleic acids ratio, the faster of the detection completion time.

Signal-to-noise (SNR) analysis was conducted on the fluorescent intensity generated by the MB-BRCA1 and SynBRCA1 hybridization for both standard 96-microwell plate hybridization and droplet hybridization. The fluorescence intensities of MB were measured before and after it hybridizes to its target ssDNA in order to characterize the signal-to-noise ratio (SNR) value of the MB by a fluorometer (Thermo Electronics, MA). 100 μl of 2 μM MB-BRCA1 was used to hybridize with 100 μl of different concentrations of target ssDNA and non-specific ssDNA (i.e., 250 nM, 500 nM, 1 μM, 2 μM of SynBRCA1, and 2 μM of SARS-hCoV-M (NS-ssDNA)). The 200 μl mixture of MB-BRCA1 and SynBRCA1 were loaded into a 96-microwell plate (Corning Life Sciences, Acton, Mass.). SNR data was also obtained using droplets 60. Table 1 reproduced below shows the compared data.

TABLE 1

| | | MB + | MB + | MB + | MB + |
|---|---|---|---|---|---|
| | MB + 2μM | 250 nM | 500 nM | 1 μm | 2 μm |
| SNR Value | NS-ssDNA | ssDNA | ssDNA | ssDNA | ssDNA |
| 96 Microwell | 1.0 | 23.8 | 52.5 | 118.0 | 158.5 |
| Droplet | 1.0 | 20.6 | 43.2 | 88.8 | 167.6 |

From the data in Table 1, it can be seen that the SNRs measured from the microfluidic system 10 are very similar to the SNRs measured by fluorometer in 96-microwell plates. However, the advantages of using the microfluidic system 10 include increased accuracy of detection for picoliter-sized volume samples. For instance, the microfluidic system 10 produced an accumulate fluorescence intensity of 2.8×10³ using multiple microdroplets 60 that had a total volume of around 333.3 nanoliters (in 10 seconds). This compares with a 200 μl sample in one microwell of a 96-microwell plate for one sample measurement. In addition, the detection concentration of target nucleic acid can be easily changed by adjusting the flow rates of the sample 32 and MB 42. In addition, it is relatively easy to get optimal mixing ratio of MB 42 and nucleic acids 32 for the highest signal-to-noise ratio by changing the flow rates. Finally, only a few microliters of sample are needed for detection analysis.

The microfluidic analysis system 10 may be operated by flowing sample material 32 along with MB 42 into respective inlets 30, and 40 of the microfluidic device 12. The carrier material (e.g., silicon oil) is also pumped or otherwise driven through the channels 20, 22 where droplets 60 are generated in the droplet generation region 24. Each droplet 60 contains MB 42 and the sample material 32. The sample material 32 may (or may not) contain the target nucleic acid sequence of interest. If the target nucleic acid sequence of interest is present in the sample material 32, then the complementary sequences will hybridize, causing the MB 42 to emit fluorescent radiation. For example, the hybridization of the MB 42 and the target nucleic acid sequence will cause the stem portion of MB 42 will separate and the fluorophor 106 will emit fluorescence. This fluorescence may then be picked up with the optical system 70 which detects fluorescence in the detection window 80 located in the mixing channel 50. The level of fluorescence that is measured may be compared against a pre-defined threshold value that sets the lower detection limit. Once the measured fluorescence exceeds this pre-defined threshold value, detection is registered. The pre-defined threshold may comprise an instantaneous fluorescence level, an average or median fluorescent level, or even a cumulative measure of fluorescence over a period of time, which may be adjusted by the user. The comparison between the pre-defined threshold and the measured fluorescence may be made by the computer 88 operatively coupled to the optical system 70 (e.g., via the controller/signal processing circuitry 86). The computer 88 contains fluorescence analysis software which may be used to identify detections of target nucleic acids of interest.

One advantage of the system 10 is the ability to amplify the fluorescent signal by obtaining images of flowing droplets 60 over a period of time. For example, the amount of fluorescent radiation emitted from a single droplet is relatively small. However, if the camera 84 is able to take a continuous exposure over an elapsed period of time, many droplets 60 will pass by, thereby amplifying the fluorescent signal. This sort of time-elapsed photography enables significant amplification of the fluorescent signal. Because the droplets 60 are virtually identical, each passing droplet 60 results in increased amplification. For example, 400 droplets 60 that are imaged by the camera will result in an amplification of the fluorescent signal of 400 times.

The microfluidic analysis system 10 is very selective and sensitive, and also is a fast gene detection platform. The device and system can be used in point-of-care diagnostic applications, food testing, environmental testing, and biowarfare detection. In addition, the system may be used for real-time or near real-time detection of mRNA in living cells.

The microfluidic analysis system 10 provides an excellent reactor platform for biological and chemical reagents. The system has excellent mixing in the generated droplets and can greatly accelerate the hybridization rate of molecular beacons and target nucleic acid sequences like target breast cancer genes. This system has capability for precise mixing of controlled volumes of reagents. Other advantages include simple sample preparation, minimal reagent contamination in channels, rapid detection, and requiring only a few microliters of samples for detection.

The method and system described herein overcomes problems in prior microfluidic devices. In traditional microfluidic devices, multiple streams are mixed in a pressure-driven laminar flow. Unfortunately, diffusion leads to dilution of the samples in the microchannels and also leads to cross-contamination of biological or chemical samples. Mixing is also a challenge in conventional microfluidic devices. The side-by-side nature of merging streams means that mixing can occur only by diffusion at the stream interface (i.e., stream striation). The current invention overcomes these limitations and produces good mixing efficiency and no reagent dispersion within the microchannel.

The present invention also vastly reduces the amount of time to detect hybridization between a target nucleic acid and a molecular beacon. The hybridization time has been reduced from at least 10 minutes to about 1 second. In other words, hybridization or the detection time has been decreased by at least 600 times. In addition to rapid detection, the present invention only needs a few microliters of sample for detection. By varying the operating parameters of the device, the size or volume of droplets can be precisely controlled. This aids for precisely quantifying fluorescent detection levels.

It should be understood that, multiple or different MBs can be used for different targets. For example, different fluorophors emitting different wavelengths of light can target different target nucleic acid sequences. In other words, the system can do multiplex detection simultaneously in a single picoliter-sized droplet 60. For instance, the system can multiplex a sample for genes, mRNA, pathogens, antibodies, and protein.

Another application of the system 10 described herein is the ability to monitor and analyze the binding kinetics between MBs 42 and a target analyte. For example, the level of detected fluorescent radiation detected along portions of the mixing channel 50 are indicative of the level or binding between the MBs 42 and the target analyte. Because the velocity of the droplets 60 within the mixing channel 50 can be determined, it is possible to quantify binding between the MBs 42 and the target analyte at particular times. This may be accomplished from initial mixing through saturation to give a complete picture of the binding kinetics.

It should be understood that the device described herein may be used in a variety of gene detection and disease diagnosis systems. For example, it can be used as a sensitive and fast detection system for genes, diseases, viruses, pathogens, proteins, cells, and cancer by using a small volume of sample.

While embodiments of the present invention have been shown and described, various modifications may be made without departing from the scope of the present invention. The invention, therefore, should not be limited, except to the following claims, and their equivalents.

What is claimed is:

1. A system for detecting the presence of a target nucleic acid sequence in a sample comprising:
a microfluidic device comprising a droplet generation region for forming a plurality of droplets, the droplet generation region being operatively coupled to a sample inlet coupled to a sample source, a molecular beacon inlet coupled to a source of molecular beacon material configured to hybridize to the target nucleic acid sequence, and a source of a carrier material on opposing sides of the droplet generation region, the microfluidic device further comprising a mixing channel coupled to the droplet generation region; and
an optical system configured to detect fluorescent emissions from droplets flowing in the mixing channel.

2. The system of claim 1, wherein the optical system comprises a source of illumination and a camera configured to capture fluorescent emissions from the droplets flowing in the mixing channel.

3. The system of claim 1, wherein the optical system comprises signal processing circuitry operatively connected to the camera.

4. The system of claim 3, further comprising a computer operatively coupled to the signal processing circuitry, the computer including computer readable instructions adapted to analyze one or more parameters of the fluorescent emissions.

5. The system of claim 1, further comprising pumps operatively connected to the sample and molecular beacon inlets.

6. The system of claim 1, wherein the carrier material is operatively connected to a pump.

7. The system of claim 6, wherein the carrier material comprises oil.

8. The system of claim 1, wherein the mixing channel has a sawtooth configuration.

9. The system of claim 1, wherein the mixing channel has a substantially straight configuration.

10. A microfluidic device comprising:
a substrate;
a first inlet disposed in the substrate and coupled to first and second channels terminating on opposing sides of a junction;
second and third inlets disposed in the substrate, the second and third inlets being coupled to respective channels terminating at an upstream side of the junction; and
a mixing channel coupled a downstream side of the junction, the mixing channel comprising a channel having a sawtooth configuration.

11. The microfluidic device of claim 10, further comprising a reservoir coupled to the mixing channel.

12. The microfluidic device of claim 10, further comprising pumps operatively coupled to the first, second, and third inlets.

13. The microfluidic device of claim 10, wherein the sawtooth configuration comprises a plurality of substantially 90° turns in the mixing channel.

14. A method of detecting the presence of a target nucleic acid sequence in a sample comprising:
providing a microfluidic device having a mixing channel coupled to an output of a droplet generation region, the droplet generation region being coupled to first and second channels adapted to contain an oil phase and first and second reactant inlets, wherein one of the first and second reactant inlets is coupled to a sample and the other of the first and second reactant inlets is coupled to a source of molecular beacon material;
flowing the first and second reactants and the oil phase into the droplet generation region so as to generate a plurality of droplets in the generation zone; and
monitoring the fluorescence level of the droplets in at least a portion of the mixing channel, wherein the fluorescence level is indicative of the presence of the target nucleic acid sequence in the sample.

15. The method of claim 14, wherein the mixing channel comprises a channel having a sawtooth configuration.

16. The method of claim 14, wherein the target nucleic acid is detected by comparing a measured fluorescence level against a threshold level and detecting the target nucleic acid if said measured fluorescence level is above the threshold level.

17. The method of claim 16, wherein the comparison of the measured fluorescence level is made by a computer operatively connected to an optical system configured to measure the fluorescence level in the droplets.

18. The method of claim 14, wherein fluorescence level is detected via a high-speed camera.

19. The method of claim 14, wherein the nucleic acid comprises DNA or RNA.

20. A method of monitoring the binding kinetics of an analyte and a molecular beacon comprising:
providing a microfluidic device having a mixing channel coupled to an output of a droplet generation region, the droplet generation region being coupled to first and second channels adapted to contain an oil phase and first and second reactant inlets, wherein one of the first and second reactant inlets is coupled to a source of analyte and the other of the first and second reactant inlets is coupled to a source of molecular beacon material;
flowing the first and second reactants and the oil phase into the droplet generation region so as to generate a plurality of droplets in the generation zone; and
monitoring the fluorescence level of the droplets in at least a portion of the mixing channel, wherein the fluorescence level at a given point in the mixing channel is indicative of the binding kinetics between the analyte and the molecular beacon.
